(19) United States
(12) Patent Application Publication
(10) Pub. No.: US 2011/0245689 A1
(43) Pub. Date: Oct. 6, 2011

(54) APPARATUS FOR DECIDING RISK OF ABNORMALITY OF SUBJECT'S THERMOREGULATION

(75) Inventors: Takuji Suzuki, Kanagawa-ken (JP); Kanako Nakayama, Kanagawa-ken (JP)

(73) Assignee: KABUSHIKI KAISHA TOSHIBA, Tokyo (JP)

(21) Appl. No.: 12/880,732

(22) Filed: Sep. 13, 2010

(30) Foreign Application Priority Data
Mar. 31, 2010 (JP) 2010-084321

(51) Int. Cl. A61B 5/0205; A61B 5/026

(52) U.S. Cl. 600/483; 600/504

(57) ABSTRACT

According to one embodiment, an apparatus for deciding a risk includes a measurement unit and an analysis unit. The measurement unit is configured to measure a physiological index of a subject' s peripheral blood flow. The analysis unit is configured to calculate a ripple appeared in the physiological index and to decide a risk of abnormality of the subject' s thermoregulation by the ripple.

### CROSS-REFERENCE TO RELATED APPLICATION

This application is based upon and claims the benefit of priority from Japanese Patent Application No. 2010-084321, filed on Mar. 31, 2010; the entire contents of which are incorporated herein by reference.

### FIELD

Embodiments described herein relate generally to an apparatus for deciding a risk of abnormality of a subject's thermoregulation by measuring a physiological index of the subject's blood flow.

### BACKGROUND

In order to prevent heat stress disorder, by measuring a temperature and a humidity, a device for displaying risk of a heat stress disorder under this environment is on the market. In this device, an index (WBGT) called as “a thermal index” is calculated from the temperature and the humidity, and a degree of the thermal index is displayed. However, under the same environment, risk to become the heat stress disorder is largely different by personal age, sex, life custom, physical condition, movement status and clothes. Accordingly, these factors need be monitored together, and such programs are executed.

In reference 1 (JP 4129477), from the thermal index, biological data, movement level and physical strength, a method for preventing the heat stress disorder is notified. In reference 2 (JP-A 2009-108451 (Kokai)), as to a worker in a special environment, the worker' s core temperature is monitored by measuring the number of heart beats, and warning for the heat stress disorder is informed to the worker. In reference 3 (JP 3762966), abnormality of a person is detected from data measured by a biological sensor and a weather sensor, and the abnormality is informed to the third party after confirming the person. In reference 4 (JP-A 2002-24957 (Kokai)), a physiological index of a thermal model of the human body is estimated by personal information and environmental information, and a risk decision for living body damage (heat stress disorder, hypothermia) is performed.

In reference 5 (JP-A 2008-241135 (Kokai)), a peripheral skin temperature is measured, and a periodic fluctuation (it is called “ripple”) thereof is measured. From the periodic fluctuation, a person' s thermal sensation (feeling such as cold or hot) is detected.

In any of the patent references 1˜4, as to the physiological index to be used, a concrete decision standard based on a physiological mechanism is not disclosed. In these references, the abnormality is decided using a body temperature (core temperature). However, when the core temperature is rising, the person has already become the heat stress disorder. Briefly, even if the abnormality is detected at this timing, the person cannot be prevented from the heat stress disorder.

In order to prevent the heat stress disorder, by quickly detecting a risk of the abnormality, the risk need be informed to the person. As to the heat stress disorder and the hypothermia, a function of the thermoregulation is broken down, and the user' s thermoregulation cannot be performed. Accordingly, in order to detect the risk as soon as possible, a first step to break down the function need be checked.

### BRIEF DESCRIPTION OF THE DRAWINGS

FIG. 1 is a block diagram of a risk decision apparatus according to a first embodiment.

FIGS. 2A-2D are graphs showing peripheral skin temperature and thermal sensation in correspondence with environmental temperature.

FIGS. 3A and 3B are schematic diagrams of risk-detection of heat stress disorder.

FIGS. 4A and 4B are flow charts of processing of risk-detection of heat stress disorder.

FIG. 5 is schematic diagrams of risk-detection of hypothermia.

FIGS. 6A and 6B are flow charts of processing of risk-decision of hypothermia.

FIG. 7 is a block diagram of a risk decision apparatus according to a second embodiment.

FIG. 8 is a display example of risk decision result according to the second embodiment.

### DETAILED DESCRIPTION

In general, according to one embodiment, an apparatus for deciding a risk includes a measurement unit and an analysis unit. The measurement unit is configured to measure a physiological index of a subject' s peripheral blood flow. The analysis unit is configured to calculate a ripple appeared in the physiological index and to decide a risk of abnormality of the subject' s thermoregulation by the ripple.

Hereinafter, embodiments will be explained by referring to the drawings. The present invention is not limited to the following embodiments.

### The First Embodiment

As to a decision apparatus of the first embodiment, a temperature measurement unit to measure a finger' s skin temperature (peripheral skin temperature) as a living body index of the subject is used.

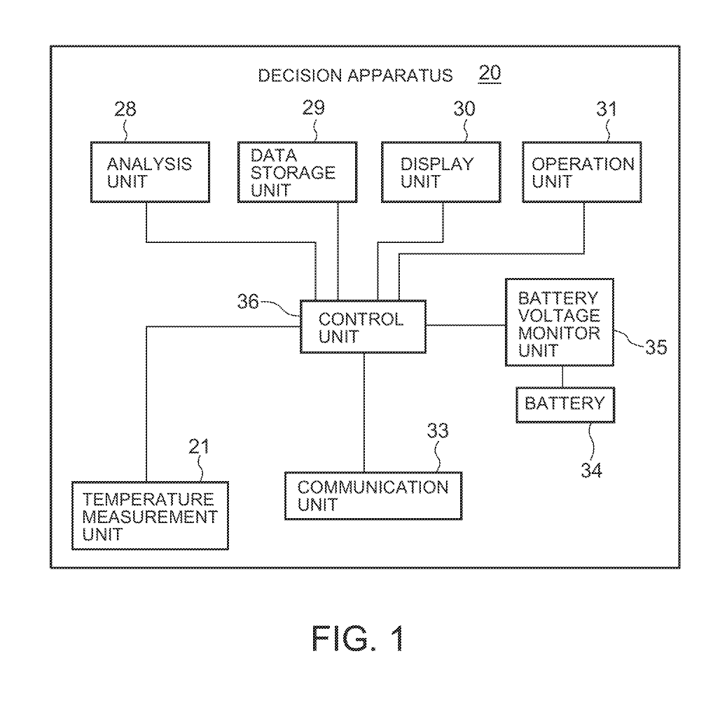

As shown in FIG. 1, the decision apparatus 20 includes a temperature measurement unit 21, an analysis unit 28, a data storage unit 29, a display unit 30, an operation unit 31, a communication unit 33, a battery 34, a battery voltage monitor unit 35, and a control unit 36.

As to the decision apparatus 20, for example, the general view is a ring shape including above-mentioned all units. Alternatively, the general view is a wristwatch shape from which the temperature measurement unit 21 (connected to a main body 20 via a cable) is attached with the finger. In case of the wristwatch shape, the temperature measurement unit 21 is located to attach with a palm side of the finger. In the temperature measurement unit 21, by using a digital temperature sensor (For example, SHT-11 manufactured by Sensiricon Inc.), the sensor chip converts a skin temperature to a digital value, and transmits the digital value to the control unit 36 by serial-communication (For example, I2C or SPI).

Above-mentioned processing may be executed as an analogue value. In this case (For example, the temperature measurement unit 21 is a thermistor), a register measurement unit (not shown in FIG. 1) converts change of a resister value to a voltage, an A/D converter (not shown in FIG. 1) converts the voltage to a digital value, and the digital value is transmitted to the control unit 36. The digital value is converted to a register value of the thermistor by using a characteristic of the register measurement unit, and the register value is converted to a temperature value by using a characteristic function between the register value and the temperature (previously set in the control unit 36).

Based on temperature data acquired by the control unit 36, the analysis unit 28 decides a risk of abnormality of thermoregulation. The data storage unit 29 stores not only the temperature data but also a risk-decision value of abnormality of thermoregulation (decided by the analysis unit 28).

The display unit 30 is a part to display a time, a skin temperature, a risk-decision result, a battery status, a memory status, and a communication status. Concretely, the display unit 30 can be composed as a LCD (Liquid Crystal Display).

The operation unit 31 equips a mode-switch (between a time mode and a measurement mode) or a push-switch to turn on a backlight.

The communication unit 33 transmits to/receives from an external apparatus. For example, when the analysis unit 28 detects a risk of abnormality of a user' s thermoregulation, the communication unit 33 sends the risk information to an external server. For example, by notifying the family or helpers (located at remote place) of the risk, a service to hurry to the user for help can be performed.

The communication unit 33 is a part to communicate data with an external apparatus such as a PC, a PDA terminal or a cellular-phone. Concretely, the communication unit 33 can be composed as a Bluetooth.

The battery 34 supplies power to all of the decision apparatus 20. The battery voltage monitor unit 35 monitors a voltage of the battery 34.

The control unit 36 controls all of the decision apparatus 20. By accepting a subject' s request and indication, the control unit 36 controls a processing request and a data flow for each unit. Concretely, by accepting the subject' s request, the control unit 36 controls ON/OFF of the power supply, start and various processing related to measurement.

Next, as to the decision apparatus 20 of the first embodiment, risk-decision processing is explained. A human' s thermoregulation system mainly includes a physiological control (such as peripheral blood flow, sweating or trembling) and a behavior control (such as change of clothes). By totally controlling them, the human always keeps a core temperature at a fixed value, and maintains his/her life.

In control of the physiological index, first, a body temperature is adjusted by controlling a peripheral blood flow. If this adjustment is difficult, reaction such as sweating or trembling appears. The peripheral blood flow is controlled by an autonomic nerve of blood vessel. When the environment becomes hot or a human performs the exercise, thermo produces from the human' s body, and a core temperature of the human rises. In order to lower the core temperature, by increasing a blood mass flown into a peripheral blood vessel, the human reacts to radiate from the peripheral to the outside.

Conversely, when the environment becomes cold and the core temperature of the human drops, in order to raise the core temperature, by decreasing or stopping a blood mass flown into a peripheral blood vessel, the human reacts not to radiate from the peripheral to the outside. By these two reactions, the body temperature is constantly kept. Accordingly, if the thermoregulation system is normal, the peripheral blood mass always changes by these two reactions. Furthermore, a skin temperature changes in accordance with fluctuation of the blood mass. Briefly, the skin temperature has ripple. A range having such status is called a thermoregulatory range by blood vessel.

Moreover, for example, when the environment becomes too hot for the human' s body to control the peripheral blood flow by this reaction, or when the exercise is performed too much for the peripheral blood flow to be controlled by this reaction, the blood vessel always remains opening, and fluctuation (ripple) of the blood mass does not occur. In some meaning, this represents breakdown of function of the thermoregulation system by blood. When this situation continues and the thermoregulation by sweating is difficult, the human will get heat stress disorder soon. Accordingly, by detecting breakdown of thermoregulation system with blood, risk of the heat stress disorder can be detected.

Furthermore, conversely, when the environment becomes too cold for the human's body to supplement thermo by thermogenesis, and when the core temperature drops even if the peripheral blood vessel remains closing, the human will get hypothermia soon. In this case, in the same way as heat stress disorder, timing when the ripple does not occur by reduction of the peripheral blood mass represents occurrence of a risk of hypothermia. Accordingly, by detecting this timing, the risk of hypothermia can be detected.

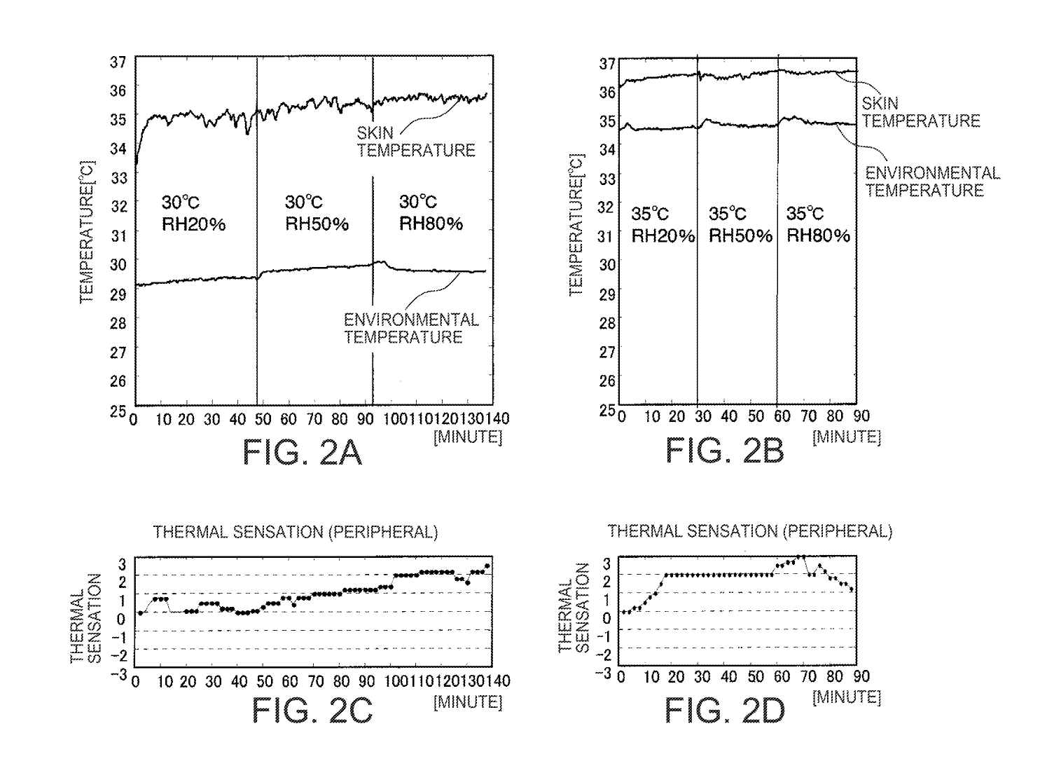

As shown in FIG. 2A, the ripple appears at environmental temperature and humidity “30° C.RH20%-50%”. However, as shown in FIG. 2B, the ripple disappears at environmental temperature and humidity “35° C. This observation represents, thermoregulation by the blood vessel is broken down at 35° C. and the blood vessel remains opening. Processing to decide a risk of abnormality of thermoregulation using this mechanism is explained.

First, a method for detecting ripple and temperature gradient is explained. As to the analysis unit 28 in the decision apparatus 20, based on measured data of skin temperature, amplitude of ripple (ripple amplitude) of skin temperature and gradient of change of skin temperature are detected. As shown in the reference 5, a frequency band of the ripple is 0.005-0.04 Hz. Accordingly, for example, a linear regression is executed within a time window (such as two minutes) including the frequency band sufficiently, and a temperature gradient is acquired from a gradient of a regression straight line. A difference between the regression straight line and the measured data is calculated, and low frequency components are eliminated from the difference. After that, a local maximum value and a local minimum value are detected from the difference, and amplitude is detected from a time difference between the local maximum value and the local minimum value. If the amplitude is above some threshold, it is decided that ripple is included in the skin temperature. In this case, a frequency of the ripple is calculated as a reciprocal number of two times of the time difference.

Moreover, as the method for detecting ripple, except for detection of the local maximum value and the local minimum value, by using frequency analysis method such as Fourier transform, estimation by AR model or wavelet transformation, a power adjacent 0.005-0.04 Hz (as a particular frequency) may be extracted.

As a first method for deciding a risk of heat stress disorder, the amplitude of ripple and the temperature gradient are compared with respective threshold (threshold 1, threshold 2). A time when both the amplitude of ripple and the temperature gradient are below the respective threshold is counted, and the counted value is set as the risk. As a second method, the temperature gradient is compared with threshold 2. If the temperature gradient is below the threshold 2, a difference between the amplitude of ripple and maximum amplitude (previously acquired) is integrated, and the integrated value is set as the risk.

Furthermore, after both the amplitude of ripple and the temperature gradient are below the respective threshold, when the skin temperature further rises, it is decided that the risk is very high.

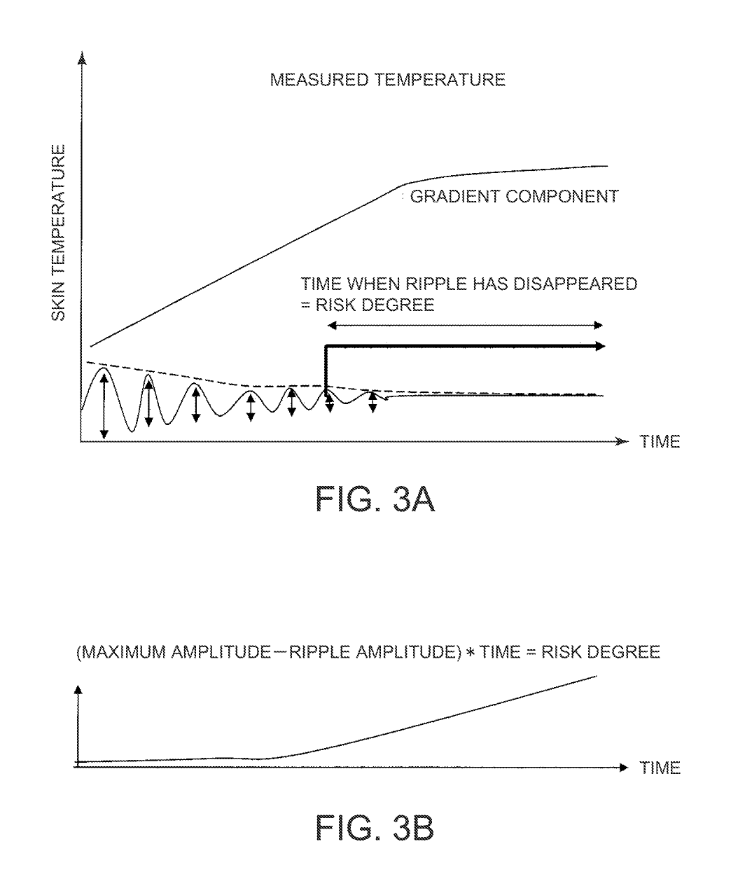

FIG. 3A shows calculation result by the first method. Briefly, change of skin temperature is separated into ripple component and gradient component, and they are represented as a graph having a horizontal time axis. In this case, by counting a time when both the amplitude of ripple and the temperature gradient are below the respective threshold, the risk is calculated. FIG. 3B shows calculation result by the second method. Briefly, a difference between the amplitude of ripple and the maximum amplitude of ripple is calculated. By integrating the difference along the horizontal time axis, the risk is calculated.

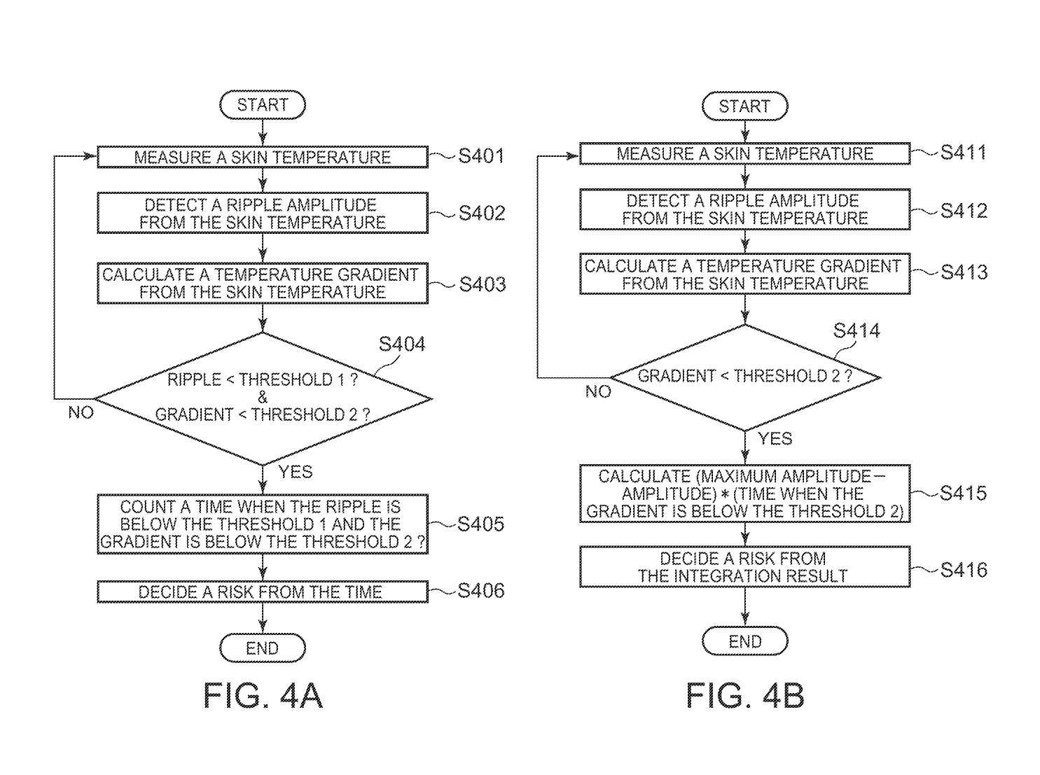

Next, processing to decide risk of heat stress disorder is explained by referring to a flow chart. FIG. 4A shows processing to decide risk by a time range not including ripple. In this case, first, a skin temperature is measured (S401). As mentioned-above, by detecting a local maximum value and a local minimum value, amplitude of ripple is detected from the skin temperature (S402). Furthermore, a temperature gradient is calculated by the linear regression (S403). The amplitude of ripple and the temperature gradient are compared with respective threshold (S404). Time when the amplitude of ripple is below threshold 1 and the temperature gradient is below threshold 2 is counted (S405).

FIG. 4B shows processing to integrate the difference between the amplitude of ripple and the maximum amplitude. In this case, first, a skin temperature is measured (S411). As mentioned-above, by detecting a local maximum value and a local minimum value, amplitude of ripple is detected from the skin temperature (S412). Furthermore, a temperature gradient is calculated by the linear regression (S413). The temperature gradient is compared with threshold 2 (S414). If the temperature gradient is below threshold 2 (Yes at S414), a difference between the amplitude of ripple and the maximum amplitude is integrated (S415), and this integrated value is set as the risk (S416).

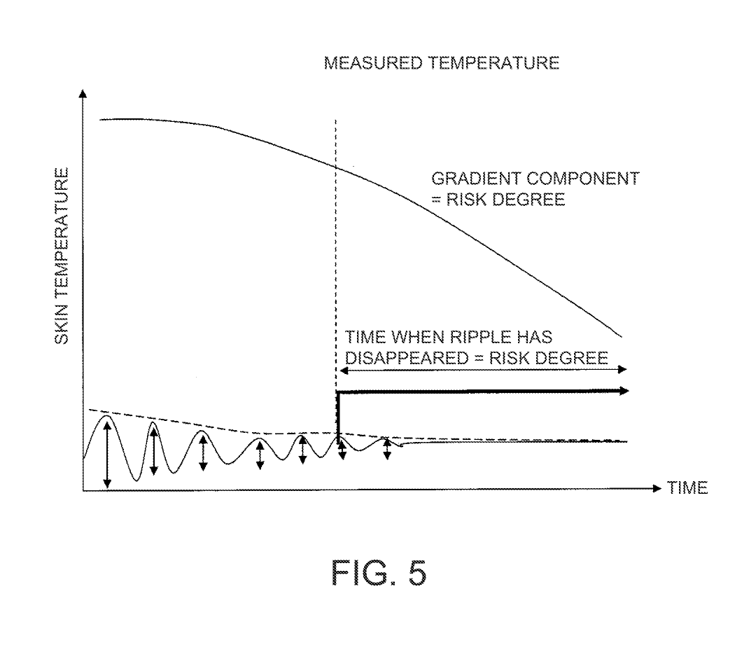

On the other hand, as a first method for deciding risk of hypothermia, a time when amplitude of ripple is below a predetermined threshold (threshold 3) is counted, and the counted value is set as the risk (S406). As a second method for detecting risk of hypothermia, when the amplitude of ripple is below threshold 3 and when the temperature gradient is larger along a minus direction, a risk degree is decided to be higher. In this case, the risk is calculated from this gradient component. The relationship between the risk degree and the gradient component (or time when ripple has disappeared) is shown in FIG. 5.

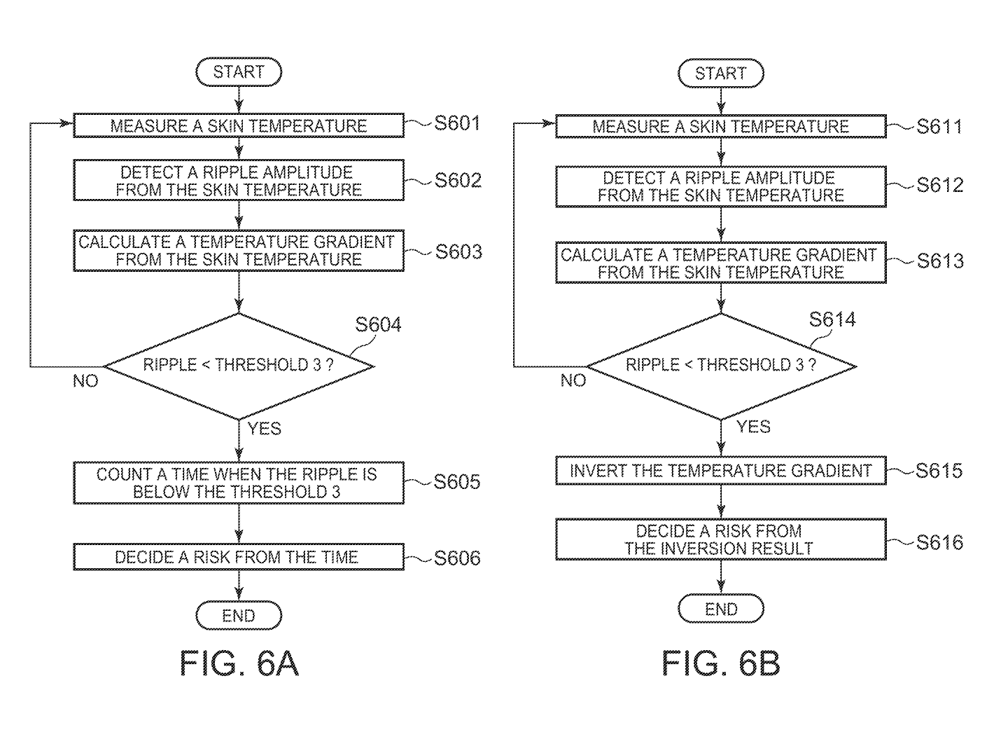

Next, processing to detect risk of hypothermia is explained by referring to a flow chart. FIG. 6A shows processing to decide risk by a time range not including ripple. In this case, first, a skin temperature is measured (S601). As mentioned-above, by detecting a local maximum value and a local minimum value, amplitude of ripple is detected from the skin temperature (S602). Furthermore, a temperature gradient is calculated by the linear regression (S603). If the amplitude of ripple is below threshold 3 (Yes at S604), time when the amplitude of ripple is below threshold 3 is counted (S605). The counted value is set as a risk of hypothermia (S606).

FIG. 6B shows processing by gradient component. In this case, first, a skin temperature is measured (S611). As mentioned-above, by detecting a local maximum value and a local minimum value, amplitude of ripple is detected from the skin temperature (S612). Furthermore, a temperature gradient is calculated by the linear regression (S613). The temperature gradient is compared with threshold 2 (S414). If the amplitude of ripple is below threshold 3 (Yes at S614), an inversion value of the temperature gradient is calculated (S615), and this inversion value is set as the risk (S616).

The risk (acquired as mentioned-above) is recorded in the data storage unit 29 by the control unit 36. Moreover, in this case, the ripple is detected from the skin temperature. However, for example, by measuring a blood flow of skin using a laser Doppler blood flowmeter, the ripple may be detected from the blood flow. In this case, the risk can be measured using the ripple.

### The Second Embodiment

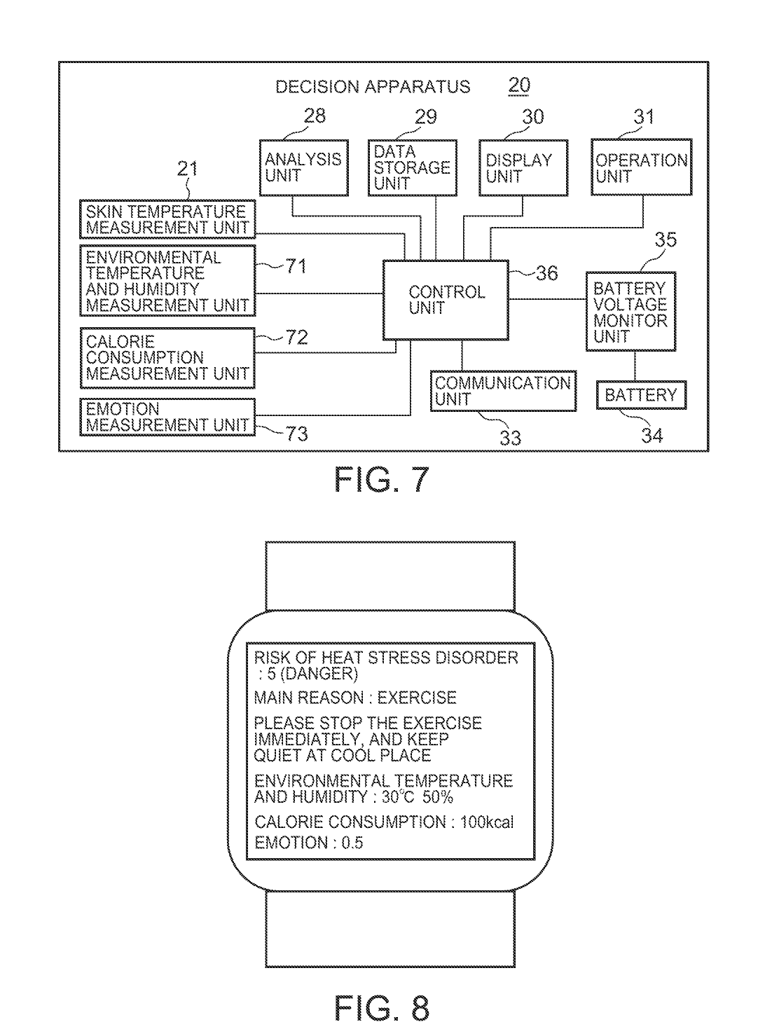

As shown in FIG. 7, in comparison with the first embodiment, at least one of an environmental temperature and humidity measurement unit 71, a calorie consumption measurement unit 72 and an emotion measurement unit 73, is added to the decision apparatus 20. As to the same unit as the first embodiment, its explanation is omitted.

A skin temperature measurement unit 21 is same as the temperature measurement unit of the first embodiment. The environmental temperature and humidity measurement unit 71 is a temperature and humidity sensor (For example, SHT-11 manufactured by Sensirion Inc.). In this sensor chip, temperature and humidity are acquired by converting to digital values, and the digital values are transmitted to the control unit 36 by serial-communication. Furthermore, the calorie consumption measurement unit 72 is an acceleration sensor having triple axes. In this sensor, scalar of triple axes of acceleration (applied to the sensor) is calculated, and a consumed calorie is calculated from a conversion table between the scalar and the consumed calorie.

Furthermore, the emotion sensor is a pulse wave sensor or a skin conductively sensor. In case of the pulse wave sensor, an interval per one pulse of pulse wave is acquired as a pulse wave interval. By analyzing frequency of the pulse wave interval, indexes “LF” (power of 0.05 Hz-0.15 Hz) and “HF” (power of 0.15 Hz-0.4 Hz) corresponding to activity of autonomic nerve are acquired. In this case, “HF” reflects activity of parasympathetic nerve, and “LF/HF” reflects activity of sympathetic nerve. If the calorie consumption (acquired by the calorie consumption measurement unit 72) is low and “LF/HF” is high, reaction of the emotion is decided to be high.

In the second embodiment, measurement is executed by at least one of the environmental temperature and humidity, the calorie consumption, and the emotion. Based on this measurement result, in the same way as the first embodiment, a risk of abnormality of thermoregulation is decided. When the risk is above a predetermined threshold, a ratio of the environmental temperature and humidity to a first reference, a ratio of the calorie consumption to a second reference, and a ratio of the emotion to a third reference, are respectively calculated. In case of heat stress disorder, one having the highest ratio is decided as a reason of risk of abnormality of thermoregulation. In case of hypothermia, one having the lowest ratio is decided as a main reason of risk of abnormality of thermoregulation. In either case, the ratio, the main reason and an advice to prevent heat stress disorder (hypothermia), are displayed via the display unit 30 as shown in FIG. 8.

As mentioned-above, in the apparatus for deciding risk of abnormality of subject's thermoregulation, status-change of thermoregulation system (different for each person) is quickly detected in daily life. By taking countermeasures to cope with heat stress disorder or hypothermia, prevention of an attack of disease can be realized.

While certain embodiments have been described, these embodiments have been presented by way of examples only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An apparatus for deciding a risk, comprising:
a measurement unit configured to measure a physiological index of a subject's peripheral blood flow; and
an analysis unit configured to calculate a ripple appeared in the physiological index, and to decide a risk of abnormality of the subject's thermoregulation by the ripple.

2. The apparatus according to claim 1, wherein
the physiological index includes a quantity of the subject' s peripheral blood flow or a value of the subject' s peripheral skin temperature.

3. The apparatus according to claim 2, further comprising at least one of
a temperature and humidity measurement unit configured to measure an environmental temperature and humidity;
a calorie consumption measurement unit configured to measure the subject's calorie consumption; and
an emotion measurement unit configured to measure the subject's emotion;
wherein the analysis unit decides the risk by a measurement result of the at least one.

4. The apparatus according to claim 3, wherein
the analysis unit decides the risk by integrating a difference between an amplitude of the ripple and a maximum amplitude of the ripple for a time when the ripple is below a predetermined threshold.

5. The apparatus according to claim 4, wherein
the measurement unit measures the subject' s peripheral skin temperature, and
the analysis unit detects a temperature gradient of the subject' s peripheral skin temperature, and decides the risk when the temperature gradient is approximately zero and when the ripple disappeared in the physiological index.
